(19) United States
(12) Patent Application Publication
(10) Pub. No.: US 2022/0172833 A1
(43) Pub. Date: Jun. 2, 2022

(54) Health System and Apparatus and Method of Use Thereof

(71) Applicant: Lindon Barrington Potter, Romford (GB)

(72) Inventor: Lindon Barrington Potter, Romford (GB)

(73) Assignee: Lindon Barrington Potter, Romford (GB)

(21) Appl. No.: 17/539,792

(22) Filed: Dec. 1, 2021

(30) Foreign Application Priority Data
Dec. 2, 2020 (GB) 2018991.6

(51) Int. Cl. G16H 40/63

(52) U.S. Cl. CPC G16H 40/63

(57) ABSTRACT

A system for collecting user data, said user data being a physical or physiological measurement from the user, said user data collected by an electronic device, characterised in that the electronic device measures and/or records any one, or any combination of, hand measurements, wrist measurements and/or arm measurements.

The present invention relates to an improved system for the provision of patient data, data verification and personalised healthcare.

The provision of healthcare data to a medical system is known. There are numerous instances whereby which up-to-date information associated with a patient's and consumer's physiological and demographic profile can facilitate remote/virtual interactions with service providers and product developers. Often there are limitations related to protection of personal information, verification of information, control, integrity, and accessibility of information. These factors often discourage individual from using such systems.

As a result, significant time and resources are expended by:
Patients attempting to provide information to health and healthcare organisations related themselves and products.
Organisations attempting to verify data provided to them by consumers.
Individuals updating data they have provided to an organisation a Individuals attempting to maintain oversight over data that they have provided to an organisation.
Organisations attempting to store and manage information (by generic medical screenings (insurance, employment, clinical trials, post approval safety studies and observational studies)) provided by individuals (product consumers, customers, subcontractors and patients).
Professionals and organisations providing support, care or oversight of individuals during emergencies and disasters (e.g. healthcare emergencies, power outages, extreme adverse weather etc.).

Furthermore, there are circumstances where it is not even feasible to expend resources to obtain, manage or transfer the above-mentioned information therefore it is not done and there are limited forms of mitigating actions that can be implemented. For example, if a consumer comments or complains about a medical product, this is usually done via telephone call. This therefore limits the amount of information that can be obtained and verified to information about the product (e.g. packaging quality, batch numbers, serial numbers and expiry dates etc.).

Furthermore, due to time and resource limitations customer service representatives are often unable to ask product/service specific questions to consumers or obtain or verify information related to an individual, (identity, age, sex, nationality, body measurements, health status etc.). Therefore, corrective, preventative and mitigating actions implemented by an organisation can be severely limited in their effectiveness and robustness.

It is therefore an aim of the present invention to provide system that addresses or improves the aforementioned problems.

It is a further aim of the present invention to produce a method for providing consumer or patient data to one or more organisations.

In a first aspect of the invention there is provided a system for collecting user data, said user data being a physical or physiological measurement from the user, said user data collected by an electronic device, characterised in that the electronic device measures and/or records any one, or any combination of, hand measurements, wrist measurements and/or arm measurements.

In a preferred embodiment the hand measurements include length and/or width measurements.

In a preferred embodiment the wrist measurements can include long axis, short axis and/or circumference measurements.

In a preferred embodiment the arm measurements include length and/or circumference measurements.

Typically, the circumference of the wrist and/or arm is calculated or predicted utilising measurements of the wrist and the arm to calculate the circumference.

Typically the electronic device is a mobile communication device. Further typically the electronic device is a smartphone, tablet and laptop.

In one embodiment the electronic device is connected to a portable electronic device, such as a smartphone, to send the user to an organisation.

In one embodiment a software application or web-based software program is utilised on the electronic device that utilises one or more cameras to record the hand, wrist and/or arm measurements.

Typically the user receives onscreen instructions and shown where to position their arms, hands or wrists within the field of view of the front facing and/or rear-facing camera. Further typically the user receives onscreen instructions as where to put their hands or wrist on the display screen of their device.

Further typically the device is utilised to accurately measure or calculate the user's arm length and/or arm circumference.

In one embodiment the camera of the electronic device is used to provide or record height and/or waist circumference measurements.

Typically the user or patient will receive on-screen instructions on the electronic device to show them how position their body relative to the device to do this. Further typically the on-screen instructions include demonstration videos and/or images.

In one embodiment the user's waist circumference to height ratio is calculated. Typically the user's waist and height data can be stored and/or triaged in accordance to the user's specifications.

In one embodiment were a user is unable to provide a waist circumference and/or height measurement, for example in cases were a user does not have measuring equipment, user's mobility is limited or when a user is in a public place, the application or program software determines hand length and a regression equation is used to predict the height of a user.

In one embodiment the application or program software determines the mid-upper arm circumference to predict a user's level of obesity or body mass index.

In a preferred embodiment the application or program software records and/or sends identification data such as date of birth, sex and nationality, place of birth. Typically photographic identification is used to verify their date of birth, sex and nationality.

Typically the software enables the user's electronic device to operate as a hardware interface for a software-based clinical assessment platform,

In one embodiment the platform is a centralised, integrated biomedical sample and clinical assessment management system platform.

Typically the data that is obtained from the communication device's sensors can be stored and/or triaged in accordance to the user's specifications. Further typically the data or measurements obtained can be sent to an organisation in accordance with the user's specifications.

In one embodiment of the invention on-screen instructions show the user what they need to do, in order to obtain their hand measurements. Typically the device records hand length, hand width, and/or wrist diameters (i.e. wrist short-axis length and/or wrist long-axis length).

In one embodiment the device records the measurements of the length of the user's longest finger and the length of the user's hand palm.

In one embodiment the device records the measurement of the user's hand width.

In one embodiment the user's hand measurements (e.g. hand width, hand length, wrist short axis length and wrist long axis length) are used to calculate the users wrist circumference. Typically the user's wrist circumference takes the shape of an oval the following equation will be used to calculate the predicted circumference of the user's wrist

(a=long axis length, b=short axis length, P=circumference of an oval).

$$P = 2 π \sqrt{\frac{{a}^{2} + {b}^{2}}{2}}$$

In one embodiment the user's calculated or measured height and calculated wrist circumference will be used to predict the user's obesity level.

Typically the relationship between mid-upper arm circumference and wrist circumference is positively linear or has a substantially linear correlation to waist circumference.

Typically the relationship between hand length and hand width have is positively linear or has a substantially linear correlation to height.

In a preferred embodiment of the invention, the measured or calculated wrist circumference is used to estimate the user's waist circumference.

In a preferred embodiment of the invention, the measured or calculated mid-upper arm circumference is used to estimate the user's waist circumference.

In a preferred embodiment the measured or calculated hand length is used to estimate the user's height.

In one embodiment the software enables the electronic device camera to provide an accurately measure of the number of breaths taken by a user in a predetermined period of time (e.g. 15 seconds, 30 seconds and 60 seconds). Typically the application or program software will then calculates the respiratory rate of a user. Further typically the user will receive or be presented with onscreen instructions to show them how to do this.

In one embodiment this respiratory data can be stored and/or triaged in accordance with the user's specifications (i.e. to another user or organisation). The data can also be used to build a health profile of the user. User's would typically also have the option of to enter their age and uploading their photographic identification (e.g. passport or driving license) to enable verification of age, date of birth, sex and nationality.

In one embodiment the software enables the camera of the electronic device to provide an image that will enables an assessment of a biological sample's integrity, management and/or logistics.

Typically the user will be able to take a photograph of a sample's label including barcode or QR codes if present.

Further typically the user can verify the details of the sample's container (for example batch number, storage conditions, expiry date, manufacturer etc.) and the details of the biological sample (for example patient identification, patient date of birth, time and date of sample collection).

In one embodiment the integrity of the sample and the container can be documented by the camera.

In one embodiment the application or program software enables the camera of the electronic device to provide an image that will capture the results and integrity of disposable diagnostic tests (e.g. body temperature, urinalysis and serology tests).

Typically the user will be able to take a photograph of a diagnostic test prior to using the test and/or take a photograph when any results are shown on the diagnostic test.

In one embodiment the application or program software is configured to enable the user to upload data obtained from devices synchronized or linked to the electronic device. For example, smart watches, heart monitors and other independent devices linked to the electronic device can be used to record data.

In one embodiment the independent devices communicate with the electronic devices wirelessly. Typically the wireless communication is Bluetooth® communication.

In one embodiment the application or program software includes a live chat function. Typically, the software will enable a user to engage in a live chat or video call with a clinician or healthcare worker and the user will have the ability to allow the data to be reviewed in real-time by said clinician or health worker. Further typically users would also have the option of to enter their age and uploading their photographic identification (e.g. passport or driving license) to enable verification of age, date of birth, sex and nationality.

Typically the system of the present invention can be applied in the following areas; observation studies, post-approval safety studies, medical screenings (e.g. employment, insurance etc.), in instances were infection control is a concern, in patient/customer support programmes (e.g. implemented by pharmaceutical companies), product quality complaints (e.g. medicinal products, nutraceuticals, foods etc.), reports of suspected adverse events or injuries associated with products, service complaints (e.g. packaging or condition complaints of product deliveries etc.), obtaining real-world data from consumers and patients, online shopping for garments and equipment that requires a user's height and/or waist size (e.g. garments, golf clubs and tools etc.), obtaining body measurements from disabled users who cannot stand for long periods of time, use wheel chairs or have mobility issues.

In a second aspect of the invention there is provided a method of for collecting user data, said user data being a physical or physiological measurement from the user, said user data collected by an electronic device, characterised in that the electronic device measures and/or records any one, or any combination of, hand measurements, wrist measurements and/or arm measurements.

Specific embodiment of the invention are now described with reference to the following figures wherein;

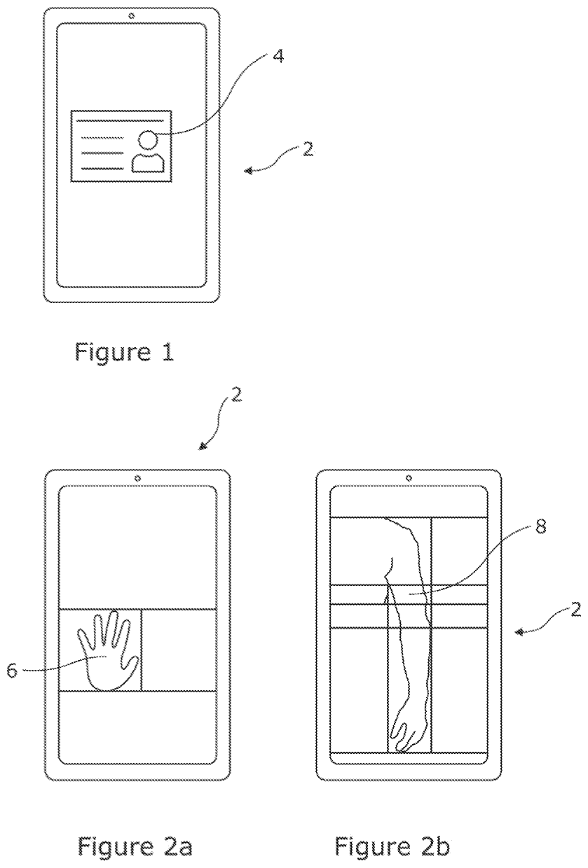

FIG. 1 shows an image of an electronic device capturing a user's identification in accordance with one embodiment of the invention;

FIGS. 2a (front viewing camera from phone/tablet PC) and 2b (rear viewing camera from phone/tablet PC) show representations of an electronic device measuring or capturing anatomical data in accordance with one aspect of the invention;

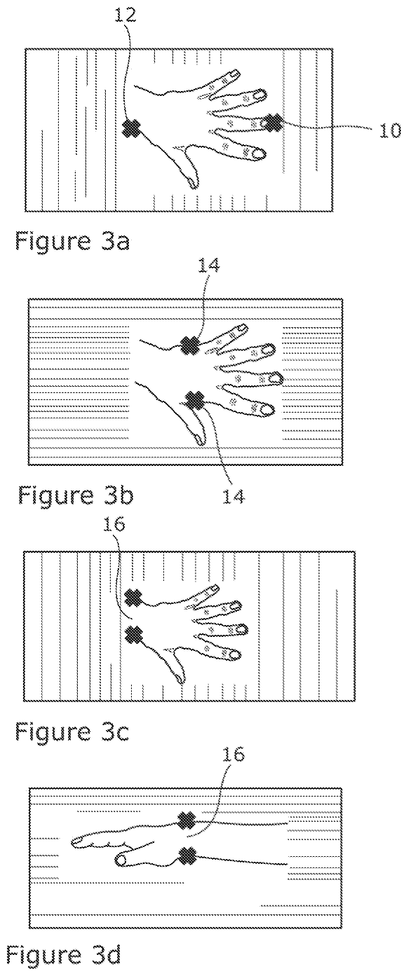

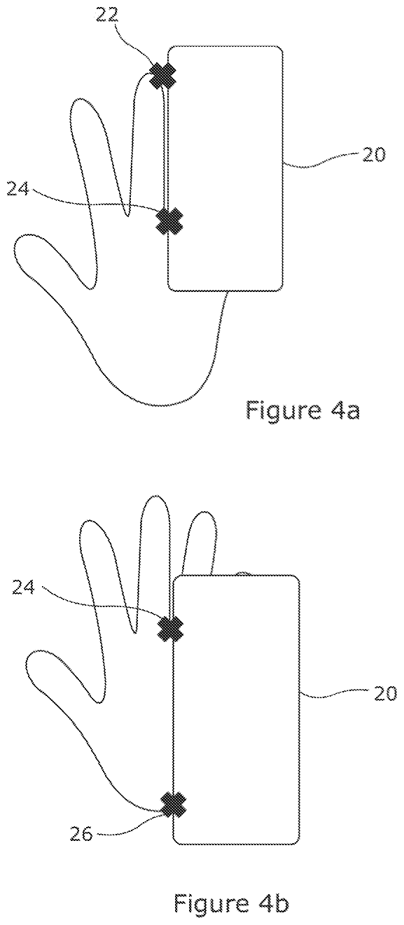

FIGS. 3a-3d (capturing hand measurements using a laptop without the use of a camera) show representations of an electronic device measuring or capturing hand or wrist anatomical data in accordance with one aspect of the invention;

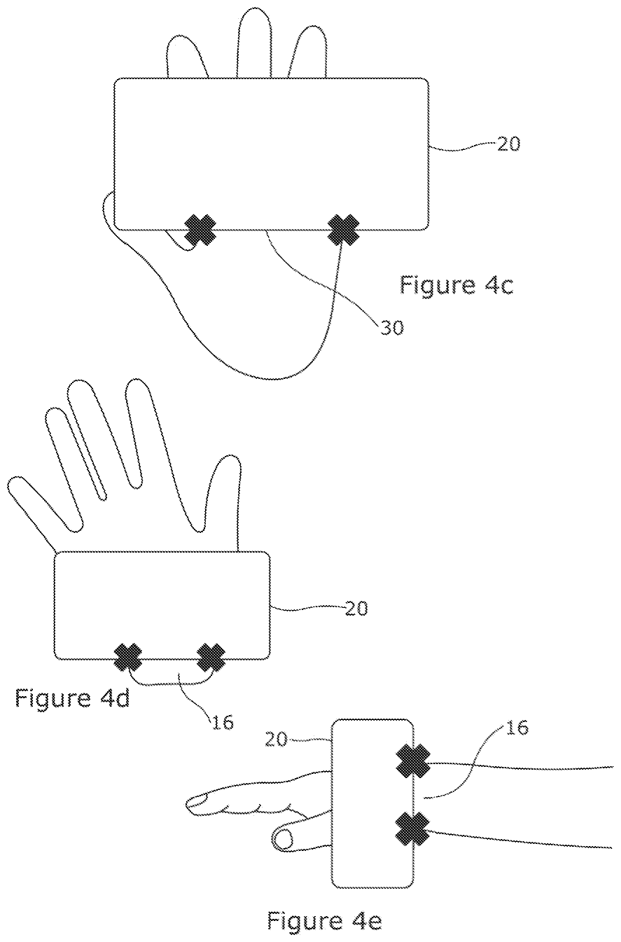

FIGS. 4a-4e (capturing hand measurements using a smart phone without the use of a camera) show representations of using the screen of an electronic device to measure or capture hand or wrist anatomical data in accordance with one aspect of the invention; and

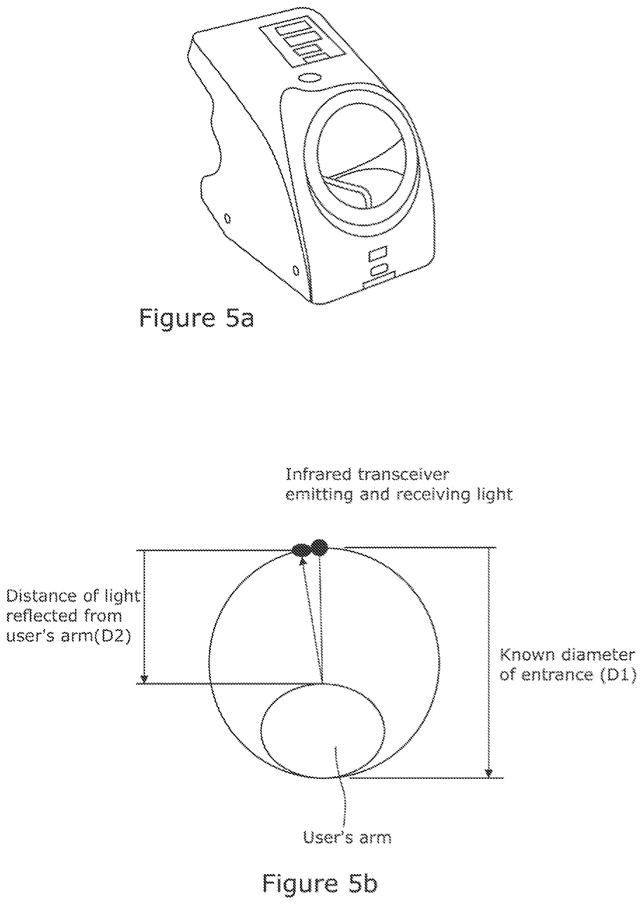

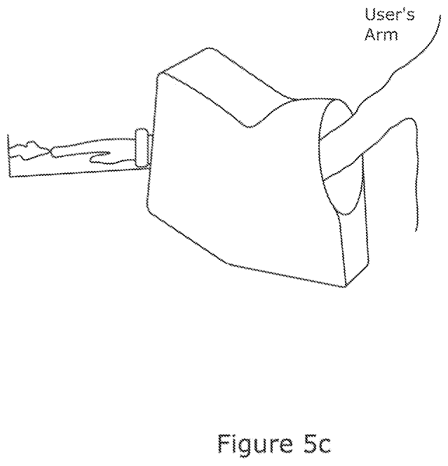

FIGS. 5a-5c show representations of a device to measure or capture hand or wrist anatomical data in accordance with one aspect of the invention.

### BACKGROUND

There are numerous instances whereby which up-to-date information associated with a patient's and consumer's physiological and demographic profile can facilitate remote/virtual interactions with service providers and product developers. Often there are limitations related to protection of personal information, verification of information, control, integrity and accessibility of information. These factors often discourage individual from using such systems.

As a result, there is a lot of time and resources expended by:
Patients attempting to provide information to organisations related themselves and products.
Organisations attempting to verify data provided to them by consumers.
Individuals updating data they have provided to an organisation.
Individuals attempting to maintain oversight over data that they have provided to an organisation.
Organisations attempting to obtaining and managing information (obtained by generic medical screenings (insurance, employment, clinical trials, post-approval safety studies and observational studies)) provided by individuals (product consumers, customers, subcontractors and patients).
Professionals and organisations providing support, care or oversight of individuals during emergencies and disasters (e.g. healthcare emergencies, power outages, extreme adverse weather etc.).

Furthermore, there are circumstances where it is not even feasible to expend resources to obtain, manage or transfer the above-mentioned information therefore it is not done and there are limited forms of mitigating actions that can be implemented.

Examples of where the proposed invention would be used:
Observation studies
Post-approval safety studies
Medical screenings (e.g. employment, insurance etc.)
In instances were infection control is a concern
In Patient/customer support programmes (e.g. implemented by pharmaceutical companies)
Product quality complaints (e.g. medicinal products, nutraceuticals, foods etc.)
Reports of suspected adverse reactions or injuries associated with products
Service complaints (e.g. packaging or condition complaints of product deliveries etc.)
Obtaining real-world data from consumers and patients.
Online shopping for garments and equipment that requires a user's height and/or waist size (e.g. trousers, golf clubs and shirts etc.).
Obtaining body measurements from disabled users who cannot stand for long periods of time, use wheel chairs or have mobility issues.

Use Case Examples

Product Complaints

If a consumer wishes to report a comment or complaint about a product (e.g. medicinal product) to the product's manufacturer and/or distributor, it is usually done through a telephone call. This limits the amount of information that can be obtained and verified about the product (e.g. packaging condition, batch numbers, serial numbers and expiry dates etc.). Furthermore, due to time and resource limitations customer service representatives are often unable to ask and verify product/service-specific questions to consumers. Furthermore, it is usually not possible for the customer service representative to obtain or verify information related to the consumer, (e.g. identity, age, sex, nationality, body measurements, level of health etc.). Therefore, any subsequent corrective, preventative and mitigating actions implemented by an organisation (in response to the consumer's product/service complaint) can be severely limited in their effectiveness and robustness.

Eligibility Assessments

In instances where and individual's body measurements may be needed quickly and efficiently (e.g., height and waist measurements etc.), in orderto verify eligibility of an individual for participation in a service, programme, clinical study and insurance policy. As well as quickly and efficiently to determine the changes to an individual's body measurements without the need for a face-to-face appointment. Additionally, when a bespoke set of questions need to be answered by an individual, based upon real-time/live data provided by an individual (e.g. age, sex, nationality, body measurements, level of health, time and date etc.).

Remote Medical Appointments

In situations where face-to-face medical appointments are unavailable or difficult to access. A telephone-based appointment (including, web-based call or video call), can be supplemented by a clinician receiving and reviewing information provided by a patient (e.g. identity, age, sex, nationality, body measurements, level of health etc.).

Customer Feedback

In situations where customer feedback is solicited by an organisation then a customer would be able to provide verifiable information about themselves (e.g. identity, age, sex, nationality, body measurements, level of health etc.).

Management of Biological Sample Collection and Logistics

Obtaining verifiable patient and/or clinical trial volunteer information quickly, enable an organisation to be able to be more efficient when it comes to determining how and when biological sample collection and processing is needed (e.g. serology, urine etc.). An information management system that is accessible by patient and/or clinical trial volunteer (using a mobile communication device) and an organisation that is responsible for managing and processing the biological sample can enable quicker and more efficient biological sample collection to occur. To prevent sample processing issues related to time delays and sample conditions.

### SUMMARY

Examples of methods are described herein.

Receiving and managing the following from a communication device:
Video and image data from a device to enable a user:
to accurately record body measurements (waist circumference and height)
to enable a user to accurately record resting respirator rate.
to allow a user to document when biological samples are being obtained and verify sample's generation, integrity, management and logistics.
to accurately record and verify the results obtained from diagnostic tests (e.g. body temperature measurement, urine analysis, serological analysis etc.) performed by a user.
to verify the details of diagnostic test (e.g. time test was taken, date test was taken, results obtained by test etc.),
to verify the identity of user by means of photograpic identification, voice identification and biometric identification.

The above functionality components will enable a communication device (e.g. smart phone) to operate as hardware (and interface) for a software-based centralised, integrated biomedical sample and clinical assessment management system platform. The data that is obtained from the communications device's sensors can be stored and/or triaged in accordance to the user's specifications (i.e. sent to another user or organisation).

To Accurately Record Body Measurements (Waist Circumference and Height)

The software will enable the camera of a communication device to provide an accurate height and waist circumference measurement In instances where a user's device camera is not available/functional the software will still enable the user's device to provide an accurate height and waist circumference measurement. The user will receive onscreen instructions to show them how to do this. The user's waist circumference to height ration will be calculated. This data can be stored and/or triaged in accordance to the user's specifications (i.e. sent to another user or organisation). The data can also be used to build a health profile of the user. The user would also have the option to enter their age and uploading their photographic identification (e.g. passport or driving license etc.) to enable verification of their age, date of birth, sex and nationality.

To Accurately Predict Height and Obesity Levels Using Arm and Hand Measurements

Additionally, in cases were a user is unable to provide a waist circumference and height measurement (for example in cases were a user's mobility is limited or when a user is in a public place). The software will determine hand length using the camera of a mobile communication device and onscreen instruction. In instances where a user's device camera is not available/functional the software will still enable the user's device to provide an accurate height and waist circumference measurement. The software will use a regression equation to predict the height of a user.). The software will also determine the Mid-upper arm circumference of user to predict a user's level of obesity (or body mass index).

User Interface Diagram for Band and Arm Measurement

The user will receive on-screen instructions on how to verify their photographic identification to capture their, date of birth, sex and nationality. FIG. 1 shows a diagram of a user's electronic device 2 capturing the details of an identification card 4, in this example a driving licence.

The user will receive onscreen instructions on where and how to position their arms and hands using the front facing and/or rear-facing camera of their mobile communication device can be utilised to accurately measure the user's arm length and arm circumference. FIG. 2a shows an example of a hand measurements 7 being captured by the device 2 and FIG. 2b shows arm measurements 8 being captured and the Mid-Upper Arm Circumference (MAC)=MUAC diameter multiplied by n.

Obtaining Hand Measurements (Web-Based)

Onscreen instructions will show the user what they need to do obtain their hand measurements (e.g. hand length, hand width, wrist diameter (short axis and axis length) etc.). The user will need to place their hand on the screen in the positions shown in FIGS. 3a-3d.
1) Hand-Length Measure

The user will need to simply double click their cursor at their longest fingertip 10 and their wrist 12, to measure their hand length (see blue crosses in FIG. 3a).
2) Hand-Width Measure

The user will need to simply double click the cursor at each side of their palms 14 for the hand width to be measured (see blue crosses in FIG. 3b).
3) Wrist Long-Axis Measure

The user will need to simply double click the cursor at each side of their wrist 16 for the long-axis of the wrist to be measured (see blue crosses in FIG. 3c).
4) Wrist Short-Axis Measure

The user will need to simply double click the cursor at each side of their wrist 16 for the short-axis of the wrist to be measured (see blue crosses in FIG. 3d).

Obtaining Band Measurements (Mobile Application-Based)

FIGS. 4a-4e show how hand measurements can be obtained using the screen of the electronic device 2.
1) Finger-Length Measurement

The user will need to touch their mobile device's screen 20 at the top of their longest fingertip 22 and the bottom of their finger 24 to measure their longest figure's length (see blue crosses in FIG. 4a).
2) Palm-Length Measurement

The user will need to touch their mobile device's screen at the bottom of their is longest finger 24 and the bottom of their palm 26 to measure their palm's length (see blue crosses in FIG. 4b).
3) Palm-Width Measurement

The user will need to touch their mobile device's screen at either side of their palm's 30 width (see blue crosses in FIG. 4c).
4) Wrist Long-Axis Measurement

The user will need to touch their mobile device's screen 20 at either side of their wrist's long-axis (see blue crosses in FIG. 4d).
5) Short-Axis Measurement

The user will need to touch their mobile device's screen 20 at either side of their wrist's short-axis (see blue crosses in FIG. 4e).

Calculations

The user's hand measurements (e.g. hand width, hand length, wrist short axis length and wrist long axis length etc.) and user's characteristics (e.g. age, gender, ethnicity, nationality etc.). Will be used to calculate:

User's Wrist Circumference Calculation

Assuming that the wrist is an oval the following equation will be used to calculate its circumference.

(a=long axis length, b=short axis length, P=circumference of an oval).

$$P = 2 π \sqrt{\frac{{a}^{2} + {b}^{2}}{2}}$$

User's Height Calculation

The hand measurements (hand length and hand width) and user's characteristics (e.g. age etc.) will be utilised to predict the height of the user.

User's Obesity Level Calculation

The user's calculated height (Stature) and calculated wrist circumference to predict the user's obesity level.

To Accurately Record Respiratory Rate

The software will enable the camera of a communication device to provide an accurately measure the number of breaths taken by a user in a predetermined period of time (e.g. 15 seconds, 30 seconds and 60 seconds). The software will then be able to calculate the respiratory rate of a user. The user will receive onscreen instructions to show them how to do this. This data can be stored and/or triaged in accordance to the user's specifications (i.e. to another user or organisation etc.). The data can also be used to build a health profile of the user. User's would also have the option of to enter their age and uploading their photographic identification (e.g. passport or driving license etc.) to enable verification of age, date of birth, sex and nationality.

To Accurately Record Cardiovascular Activity e.g. Heart Rate (HR)

The software will enable the camera of a communication device to provide an accurately measure a user's heart rate by placing the fingertips on the camera for a predetermined period of time (e.g. 15 seconds, 30 seconds and 60 seconds). The software will then be able to calculate the heart rate of a user. The user will receive onscreen instructions to show them how to do this. This data can be stored and/or triaged in accordance to the user's specifications (i.e. to another user or organisation). The data can also be used to build a health profile of the user. User's would also have the option of to enter their age and uploading their photographic identification (e.g. passport or driving license etc.) to enable verification of age, date of birth, sex and nationality.

To Document When Biological Samples are Being Obtained and Verify Sample'S Generation, Integrity, Management and Logistics

The software will enable the camera of a communication device to provide an image that will enable an assessment of a biological sample's integrity, management and logistics. The user will be able to take a photograph of a sample's label (e.g. including its details, barcode or QR code etc.). To verify the details of the simple's container (e.g. batch number, storage conditions, expiry date and manufacturer etc.) and the details of the biological sample (e.g. patient identification, patient date of birth, time and date of sample collection etc.). The integrity of the sample and the container will also be documented by the camera. If a user utilises a centrifuge as part of sample processing, then the centrifuge's label details are captured on the camera of the device (e.g. including its details, barcode or QR code, manufacturer, equipment details, qualification/validation status etc.). When a biological sample is placed in and out of a temperature-controlled container then the software will allow the user to capture the details contained on the label of a temperature-controlled container's label (including its details, barcode or QR code, batch number, storage conditions, qualification/validation status etc.). The software will enable the user to store and upload the date and time that a sample is moved inside and outside of a temperature-controlled container's. This data can be stored and/or triaged in accordance to the user's specifications (i.e. to another user or organisation etc.). The data can be added to a user's profile.

To Accurately Record and Verify the Result Obtained from Disposable Diagnostic Tests

The software will enable the camera of a communication device to provide an image that will capture the results and integrity of disposable diagnostic tests (e.g. body temperature, urinalysis and serology tests). The user will be able to take a photograph of a diagnostic test prior to using the test, take a photograph when results are shown on the diagnostic test. The user will receive onscreen instructions to show them how to do this. This data can be stored and/or triaged in accordance to the user's specifications (i.e. to another user or organisation etc.). The data can also be used to build a health profile of the user. User's would also have the option of to enter their age and uploading their photographic identification (e.g. passport or driving license etc.) to enable verification of age, date of birth, sex and nationality.

Obtaining Data from Additional Devices

The software will enable the user to upload data obtained from devices synchronized to the user's communication (e.g. smart watches, medical device etc.). The data can also be used to build a health profile of the user. User's would also have the option of to enter their age and uploading their photographic identification (e.g. passport or driving license) to enable verification of age, date of birth, sex and nationality.

Live Chat with a Clinician

The software will enable a user to engage with a live chat with a clinician (e.g. nurse or doctor etc.) and the user will have the ability to allow the data be review in real-time by a clinician. User's would also have the option of to enter their age and uploading their photographic identification (e.g. passport or driving license etc.) to enable verification of age, date of birth, sex and nationality.

Chat with a Clinician Chatbot

The software will enable a user to engage with a messaging chat with a ‘clinician chatbot’ to ask a set of pre-determined question (not for a clinical assessment).

Bespoke Assessment Triage of a User

The software will allow a user to scan barcodes (e.g. matrix 2D barcodes QR codes) and linear barcodes) located on advertisements, print media (e.g. magazines etc.), screens/monitors and upon packaging (e.g. food packaging and medicinal product packaging etc.). Upon the scanning of a barcode using the software application the software will provide the user with a bespoke questionnaire that will enable the user to use their communication device to capture the following information

The most unique elements of the proposed invention are as follows
Is the utilisation of hand and sex and age information from photographic identification to predict an individual's height.
Is the utilisation of arm measurements, hand measurements, wrist measurements and verifiable demographic information (e.g. sex and age information etc.), from photographic identification to predict an individual's obesity level (i.e. predicting waist to height ratio and predicting body mass index).
Upon completion of a bespoke questionnaire which may include the user providing their body measurements and vital sign measurements (using the methods described within this document (i.e. the software platform)) a unique barcode (e.g. a QR code etc.) will be sent to a user's mobile communication device. This will allow the unique barcode (e.g. a QR code etc.) to be scanned by a courier or clinician to:
Assist in the verification, management and transportation of biological samples.
Capture the time and date of sample collection.

To analyse relationships between the processing and analysis of biological samples and the information provided by a software user.

Hardware Application

A Waiting Room Blood Pressure Monitor and/or Health-Check Kiosk

FIG. 5a shows an image of a typical waiting room blood pressure cuff. The entrance for the arm will have a simple emitter and receiver (in this example an infrared transceiver) fitted. This transceiver will be able to measure the diameter of the arm as shown in FIG. 5b.

FIGS. 5b and 5c shows the mid-upper arm circumference (MUAC) will equal D1 minus D2. MUAC can be used to calculate a BMI and predict waist circumference.

The blood pressure monitor will calculate the height of the user by measuring the size of the users' hand.

User's hand, the had rest will have visible markings and grooves, to assist the user in positioning their hand. The transceiver at the top of the hand rest will measure the length of the hand by first calculating the space between the user's fingertips and transceiver (D1)

The subtracting it from the known distance where base of the user's wrist is placed (D1).

D1 minus D2 equals the length of the user's hands.

The user's hand length can be used to calculate the user's height.

1. A system for collecting user data, said user data being a physical or physiological measurement from the user, said user data collected by an electronic device, characterised in that the electronic device measures and/or records any one, or any combination of, hand measurements, wrist measurements and/or arm measurements.

2. A system according to claim 1 wherein the hand measurements include length and/or width measurements.

3. A system according to claim 1 wherein the wrist measurements can include long axis, short axis and/or circumference measurements.

4. A system according to claim 1 wherein the arm measurements include length and/or circumference measurements.

5. A system according to claim 1 wherein the wrist and/or arm measurement is calculated or predicted utilising measurements of the wrist and the arm to calculate the circumference.

6. A system according to claim 1 wherein the electronic device is a mobile communication device.

7. A system according to claim 6 wherein the electronic device is a smartphone, tablet and laptop.

8. A system according to claim 1 wherein the electronic device is connected to a portable electronic device, such as a smartphone, to send the user data to an organisation.

9. A system according to claim 1 wherein a software application or web-based software program is utilised on the electronic device that utilises one or more cameras to record the hand, wrist and/or arm measurements.

10. A system according to claim 9 wherein the user receives onscreen instructions and shown where to position their arms, hands or wrists within the field of view of the front facing and/or rear-facing camera.

11. A system according to claim 1 wherein the user receives onscreen instructions as where to put their hands or wrist on the display screen of their device.

12. A system according to claim 1 wherein the device is utilised to accurately measure or calculate the user's arm length and/or arm circumference.

13. A system according to claim 1 wherein the camera of the electronic device is used to provide or record height and/or waist circumference measurements.

14. A system according to claim 13 wherein the user or patient will receive on-screen is instructions on the electronic device to show them how position their body relative to the device.

15. A system according to claim 13 wherein the user's waist circumference to height ratio is calculated.

16. A system according to claim 15 wherein the user's waist and height data can be stored and/or triaged in accordance to the user's specifications.

17. A system according to claim 13 wherein if a user is unable to provide a waist circumference and/or height measurement the application or program software determines hand length and a regression equation is used to predict the height of a user.

18. A system according to claim 9 wherein the application or program software determines the mid-upper arm circumference to predict a user's level of obesity or body mass index.

19. A system according to claim 18 wherein the application or program software records and/or sends identification data such as date of birth, sex and nationality, place of birth.

20. A system according to claim 19 wherein photographic identification is used to verify their date of birth, sex and nationality.

21. A system according to claim 9 wherein the software enables the user's electronic device to operate as a hardware interface for a software-based clinical assessment platform, wherein the platform is a centralised, integrated biomedical sample and clinical assessment management system platform.
